(19) United States
(12) Patent Application Publication
(10) Pub. No.: US 2026/0008984 A1
(43) Pub. Date: Jan. 8, 2026

(54) BIOREACTOR FOR HOLDING A FLEXIBLE CONTAINER COMPRISING GEOMETRY ADAPTATION MEANS

(71) Applicant: Applikon Biotechnology B.V., Delft (NL)

(72) Inventors: Jeroen Johannes Theodorus KERKLAAN, Delft (NL); Wilbert Timotheüs WALVOORT, Delft (NL)

(21) Appl. No.: 19/326,420

(22) Filed: Sep. 11, 2025

Related U.S. Application Data
(63) Continuation of application No. PCT/NL2024/050078, filed on Feb. 15, 2024.

(30) Foreign Application Priority Data
Mar. 15, 2023 (NL) 2034342

(51) Int. Cl. C12M 1/00; C12M 3/00

(52) U.S. Cl. CPC C12M 23/14; C12M 23/26; C12M 23/28; C12M 23/48

(57) ABSTRACT

The disclosure relates to a bioreactor (1), comprising: a flexible container holder (2) for holding a flexible container (3), having a bottom wall (4) for supporting the flexible container and a circumferential wall (5), extending along a longitudinal direction (X) of the flexible container holder; a mounting portion (6) for releasably mounting the flexible container in the flexible container holder; and geometry adaptation means (7) configured for adapting the geometry of the flexible container with respect to the flexible container holder, wherein the geometry adaptation means (7) are configured for providing the flexible container (3) with a flexible container geometry similar to a rigid multi-use bioreactor geometry during use.

### CROSS-REFERENCE TO RELATED APPLICATIONS

This application is a continuation under 35 U.S.C. § 120 of International Application No. PCT/NL2024/050078, filed Feb. 15, 2024, which claims priority to Netherlands Application No. NL 2034342, filed Mar. 15, 2023, under 35 U.S.C. § 119(a). Each of the above-referenced patent applications is incorporated by reference in its entirety.

### FIELD

The present disclosure relates to a bioreactor for holding a flexible container, as well as a method for operating such a flexible container in such a bioreactor.

### BACKGROUND

A problem with existing bioreactors is that it is often difficult for an end-user (i.e. a biologist) to translate bioprocess settings from traditional bioreactors to “single-use” bioreactors, i.e. a bioreactor having a flexible container configured for single use.

An object of the present disclosure is thus to provide an improved bioreactor, wherein bioprocess settings can be more easily translated from traditional bioreactors to single-use bioreactors.

US 2020/131463 A1 discloses a bioreactor support structure configured to be used with containers having different designs.

US 2022/0235304 A1 discloses a bioprocessing system and a component management apparatus for such a bioprocessing system.

### SUMMARY

According to the present disclosure, a bioreactor is provided, comprising:
a flexible container holder for holding a flexible container, having a bottom wall for supporting the flexible container and a circumferential wall, extending along a longitudinal direction of the flexible container holder;
a mounting portion for releasably mounting the flexible container in the flexible container holder; and
geometry adaptation means configured for adapting the geometry of the flexible container with respect to the flexible container holder,
wherein the geometry adaptation means are configured for providing the flexible container with a flexible container geometry similar to a rigid multi-use bioreactor geometry during use. The Applicant has shown the insight that the geometry of a bioreactor often greatly influences the bioprocess, such as mixing times, required agitation speeds, kla's, gas uptake rates, temperature control, pressure distribution, homogeneity, and hence cell density, viability, and production (of proteins, monoclonal antibodies, et cetera). This particularly holds for single-use (e.g. production) bioreactors, i.e. bioreactors with a single-use flexible container, such as a bag, which often have geometries different from traditional bioreactors, such as a square cross-section, flat bottom and/or different height-to-diameter ratios. By providing geometry adaptation means, the geometry of the flexible container (such as a bag) with respect to the flexible container holder, can be advantageously influenced to provide improved bioprocess results with single-use bioreactors.

By providing a single-use bioreactor with such geometry adaptation means, the geometry of a rigid multi-use bioreactor can be “imitated” or “mimicked”. The Applicant has found that by using such geometry adaptation means, with a single-use bioreactor bioprocess results can be achieved that are comparable to bioprocess results obtained with a rigid multi-use bioreactor with similar geometry. “(Rigid) multi-use bioreactor (bottom) portion geometry” therein e.g. refers to a multi-use bioreactor having a traditional height-to-diameter ratio, a dish-shaped bottom portion, a hemispherical bottom portion, et cetera.

A flexible container holder with geometry adaptation means may also advantageously provide better support to the flexible container, lowering the chance of ruptures. Additionally, by increasing the contact area between the flexible container holder and the flexible container, heat transfer and hence temperature control, is improved. Furthermore, drainability of the flexible container, for instance through a bottom port, is improved, making it easier to empty the flexible container.

In the context of the present disclosure, “geometry adaption means” are broadly considered as means to influence the outer contours or external shape of the flexible container with respect to the flexible container holder when the flexible container is arranged in a multi-use (normally rigid) flexible container holder in a filled state. It should furthermore again be noted that “single-use” refers to the disposability of the flexible container—in economic terms meaning that the price of the part is outweighed by the process risks of cleaning and reusing—not necessarily of the flexible container holder, which is often (configured to be) “multi-use”.

“Geometry adaptation” therein means a change in the external shape or geometry of the flexible container when the flexible container is arranged in a multi-use flexible container holder in a filled state, compared to the external shape or geometry of the flexible container when arranged in a multi-use container flexible holder when such geometry adaptation means are not present.

Geometry adaptation may be achieved in particular by exerting pressure on the flexible container, such as an outer surface thereof, for instance a circumferential surface, a bottom surface and/or a top surface, to influence the geometry of the flexible container with respect to the flexible container holder in a filled state. Such means may comprise external/separate “objects”, such as inserts, e.g. positioned between the flexible container holder and the flexible container. Such means may also comprise positioning means, such as height-adjustment means, for positioning the flexible container with respect to, in particular against, the flexible container holder, such that the external shape of the flexible container can be influenced. The flexible container holder itself may then exert pressure on the flexible container to influence the geometry of the flexible container with respect to the flexible container holder together with the positioning means.

For sake of clarity it should be understood that “flexible container holder” should be interpreted as a “container holder for a flexible container”. The (multi-use) container holder itself is usually not flexible, but rigid.

It should be noted that US 2018/0010082 A1 and US 2021/0002597 A1 disclose a single-use bioreactor, including a bioprocess container, a shell, an agitator, a sparger, a gas filter inlet port for the sparger(s), a headspace overlay, a fill port, a harvest port, a sample port, and a probe. A controller may monitor and control one or more parameters associated with the single-use bioreactor. The shell fully encompasses/encloses the (flexible) bioprocess container and basically “forces” the bioprocess container into a certain shape, like a corsage. The pre-formed shell thus offers no geometry adaptation or adjustment after production of the shell, whereas the present invention does provide for geometry adaptation of the flexible container after production of the shell (i.e. the flexible container holder).

US 2020/131463 A1 discloses a bioreactor support structure with a spacer to be coupled with the inside of the support structure of the bioreactor at variable vertical positions, such that the vertical position of the impeller is adjustable with respect to a bioreactor bag. This is different from the present disclosure, which seeks to provide a flexible container with a flexible container geometry similar to a rigid multi-use bioreactor geometry in order to translate bioprocess settings from traditional bioreactors to single-use bioreactors with a flexible container configured for single use.

US 2022/0235304 A1 discloses a bioprocessing system and a component management apparatus for such a bioprocessing system, wherein baffles with a triangular cross-section are applied on an interior sidewall of a bioreactor to inwardly bias a disposable bioreactor bag. The flexible bag is inserted in the bioreactor vessel until the bag is supported by an angled impeller base plate provided to facilitate mating of an agitator and a magnetic drive assembly beneath the vessel. This is again different from the present disclosure, wherein a flexible container with a flexible container geometry similar to a rigid multi-use bioreactor geometry is provided in order to translate bioprocess settings from traditional bioreactors to single-use bioreactors with a flexible container.

An embodiment relates to an aforementioned bioreactor, wherein the geometry adaptation means are configured for providing a flexible container bottom portion of the flexible container with a flexible container bottom portion geometry similar to a rigid multi-use bioreactor bottom portion geometry during use. The Applicant has shown the insight that in particular the geometry of the flexible container bottom portion influences the bioprocess results, due to the influence of the flexible container bottom portion geometry on e.g. flow patterns within the bioreactor (i.e. within the flexible container).

An embodiment relates to an aforementioned bioreactor, wherein the geometry adaptation means comprise one or more inserts arranged in the flexible container holder, between the flexible container holder and the flexible container, for providing the flexible container with a flexible container geometry similar to a rigid multi-use bioreactor geometry. The inserts or inlays essentially exert pressure on the flexible container, in particular an outer surface thereof, such as a bottom surface, to obtain a flexible container geometry similar to a rigid multi-use bioreactor geometry.

An embodiment (thus) relates to an aforementioned bioreactor, wherein the geometry adaptation means comprise one or more inserts arranged on the bottom wall, between the bottom wall and the flexible bottom portion of the flexible container, for providing the flexible container bottom portion with a flexible container bottom portion geometry similar to a rigid multi-use bioreactor bottom portion geometry during use. The one or more inserts arranged on the bottom wall may for instance provide the flexible container bottom portion with a flat, dish-shaped or hemispherical bottom portion geometry.

An embodiment relates to an aforementioned bioreactor, wherein the geometry adaptation means are configured for providing the flexible container with a flat or dish-shaped flexible container bottom portion, preferably having a flexible container geometry defined by:

$${H}_{w o r k} / {D}_{w o r k} = 2. - 2.5 , preferably 2.1 - 2.3 , and / or$$

$${V}_{w o r k} / {V}_{total} = 0.6 - 0.9 , preferably 0.6 - 0.8 , more preferably 0.65 - 0.7 ,$$

wherein:
H_{work}=working height of the bioreactor
D_{work}=working diameter of the bioreactor
V_{work}=working volume of the bioreactor
Vₜₒₜₐₗ=total volume of the bioreactor

An embodiment relates to an aforementioned bioreactor, wherein the geometry adaptation means are configured for providing the flexible container with a flat or dish-shaped flexible container bottom portion, preferably having a flexible container bottom portion geometry defined by:

$$R = {D}_{u} , and / or$$

$${D}_{i} = {D}_{u} - 2 * {t}_{wall} , and / or$$

$$r = a * {D}_{u} , wherein a = 0.05 - 0.2 , and / or$$

$$h = b * {t}_{wall} , wherein b = 3 - 4 , and / or$$

$${h}_{1} = c * {D}_{u} , wherein c = 0.1 - 0.3 , and / or$$

$${H}_{b} = d * {D}_{u} + h , wherein d = 0.1 - 0.3 , and / or$$

$${V}_{b} = e * {D}_{i}^{3} , wherein e = 0.05 - 0.2$$

wherein:
R=radius of curvature of an inner portion of the flexible container bottom portion
Dᵢ=internal diameter of the bioreactor
Dᵤ=external diameter of the bioreactor
r=radius of curvature of an outer portion of the flexible container bottom portion
h₁=height of the flexible container bottom portion
V_{b}=volume of the flexible container bottom portion
t_{wall}=wall thickness of the bioreactor

The abovementioned geometry advantageously provides the flexible container flexible container bottom portion with a bottom portion geometry similar to a flat or dish-shaped bottom portion of a traditional bioreactor.

An embodiment relates to an aforementioned bioreactor, wherein the geometry adaptation means are configured for providing the flexible container with a hemispherical flexible container bottom portion, preferably having a flexible container geometry defined by:

$${H}_{w o r k} / {D}_{w o r k} = 2. - 2.5 , preferably 2.1 - 2.3 , and / or$$

$${V}_{w o r k} / {V}_{total} = 0.6 - 0.9 , preferably 0.6 - 0.8 , more preferably 0.65 - 0.7 ,$$

wherein:
H_{work}=working height of the bioreactor
D_{work}=working diameter of the bioreactor
V_{work}=working volume of the bioreactor
Vₜₒₜₐₗ=total volume of the bioreactor

The abovementioned geometry advantageously provides the flexible container flexible container bottom portion with a bottom portion geometry similar to a hemispherical or dome-shaped bottom portion of a traditional bioreactor.

An embodiment relates to an aforementioned bioreactor, wherein the geometry adaptation means comprise height-adjustment means arranged at the mounting portion, for during use adjusting a height of the flexible container in the flexible container holder. The height-adjustment means may be used to influence the geometry of the flexible container, for instance to influence the shape of the flexible container bottom portion. The height-adjustment means may be used to lower or lift the flexible container, in particular during or after filling. Thus, the pressure exerted by the bottom wall of the flexible container holder on the flexible container bottom portion—and consequently the shape of the flexible container bottom portion—may be adjusted or regulated.

An embodiment relates to an aforementioned bioreactor, wherein the flexible container is a container for a bioreaction.

An embodiment relates to an aforementioned bioreactor, wherein the flexible container is configured for single use.

An embodiment relates to an aforementioned bioreactor, wherein the flexible container comprises a bag.

An embodiment relates to an aforementioned bioreactor, wherein the flexible container holder is configured for multi-use.

An embodiment relates to an aforementioned bioreactor, comprising a flexible container arranged in the flexible container holder, wherein the mounting portion releasably mounts the flexible container in the flexible container holder.

Another aspect of the disclosure concerns a method for operating a flexible container in a flexible container holder of an aforementioned bioreactor, comprising the step of:
releasably mounting a flexible container in the flexible container holder with the mounting portion; and, optionally:
filling the flexible container with a bioreactor fluid.

An embodiment relates to an aforementioned method, comprising the step of:
arranging the geometry adaptation means comprising the one or more inserts in the flexible container holder, such that the one or more inserts are arranged between the flexible container holder and the flexible container during use, in particular for providing the flexible container with a flexible container geometry similar to a rigid multi-use bioreactor geometry.

An embodiment relates to an aforementioned method, comprising the step of:
adjusting the height of the flexible container in the flexible container holder with the height-adjustment means during filling, in particular to provide the flexible container with a wrinkle-free flexible container bottom portion. Reducing wrinkles lowers the chance of cells getting stuck and accumulating, thereby unintentionally differentiating.

An embodiment relates to an aforementioned method, comprising the step of:
adjusting the height of the flexible container in the flexible container holder with the height-adjustment means after filling, in particular to influence headspace volume and headspace geometry.

The Applicant submits that, in order to obtain an even better fit between the flexible container holder and the flexible container bottom portion, preferably the flexible container—which often has a flat bottom to be arranged on a flat bottom wall of a flexible container holder—is at least partially redesigned to (further) approximate (the bottom portion of) rigid multi-use bioreactor geometry. The flexible container itself may thus have a flat, (partially) dish-shaped or hemispherical bottom portion. Thus, rigid multi-use bioreactor geometry is even better “mimicked”, support improves, and wrinkles are even further reduced.

### BRIEF DESCRIPTION OF THE DRAWINGS

The disclosure will be explained in more detail below, with reference to illustrative embodiments shown in the drawings. Therein:

FIG. 1 shows an example embodiment of a bioreactor with a flexible container holder and a flexible container arranged therein;

FIG. 2 shows an example embodiment of a bioreactor with a flexible container holder and a flexible container arranged therein, such as the example embodiment of the bioreactor of FIG. 1;

FIG. 3 shows a perspective view of an example embodiment of a bioreactor with a flexible container holder and a flexible container arranged therein, such as the example embodiments of FIGS. 1 and 2;

FIG. 4 shows a perspective view of an example embodiment of a bioreactor with a flexible container holder, comprising geometry adaptation means in the form of inserts, without the flexible container arranged in the flexible container holder;

FIG. 5 shows a perspective view of an example embodiment of geometry adaptation means in the form of inserts, such as the inserts shown in FIG. 4;

FIG. 6 shows a schematic, cross-sectional view of a flexible container being provided with a flat/dish-shaped flexible bottom portion by using geometry adaptation means in the form of inserts;

FIG. 7 shows a schematic, cross-sectional view of a flexible container being provided with a hemispherical flexible bottom portion by using geometry adaptation means in the form of inserts;

FIG. 8 shows a graph comparing viable cell density (VCD) of a cultivation in a single-use bioreactor (SUB) provided with geometry adaptation means according to the present disclosure and a cultivation in a multi-use bioreactor;

FIG. 9 shows a graph comparing cell viability of a cultivation in a single-use bioreactor (SUB), provided with geometry adaptation means according to the present disclosure, and a cultivation in a multi-use bioreactor; and

FIG. 10 shows the bioreactor shape of the bioreactor as used with the experiments of FIGS. 8 and 9.

### DETAILED DESCRIPTION

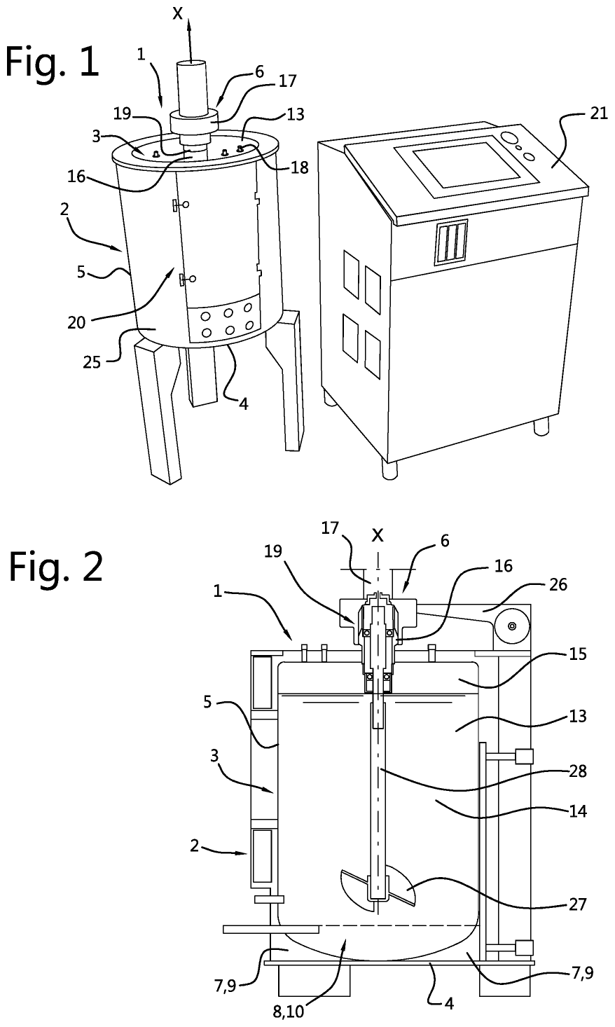

FIG. 1 shows an example embodiment of a bioreactor 1 with a flexible container holder 2, such as a bioreactor bag holder 2, comprising a drive shaft system and a flexible container 3, for single-use, i.e. to be disposed of after a single use session, such as a bioreactor bag 13 as shown in FIG. 1. The flexible container 3 may be made of a liquid impermeable material, such as polyethylene. In contrast to the flexible container 3, the flexible container holder 2 is configured for multi-use. The flexible container holder 2 may be a commonly available, rigid, multi-use flexible container holder 2. The flexible container 3 may, however, also be comprised by (not shown) a media and feed preparation system, a seed bioreactor, a hold vessel, a buffer preparation system, a mixer, a rocking bioreactor, et cetera—basically any mixing system wherein a single-use flexible container 3 can be used. As mentioned before, please note that the expression “flexible” in “flexible container holder 2” relates to the flexibility (such as foldability) of the flexible container 3, not of the container holder 2, which is usually rigid. The flexible container holder 2 and/or the flexible container 3 as shown in FIG. 1 may be configured for an operational/work volume (V_{work}) of 1-10.000 1, preferably 10-5.000 1, more preferably 50-3.000 1, such as 40-601. The flexible container holder 2 is configured for holding the flexible container 3 inside an enclosure 25, for instance a rigid enclosure 25, such as a cylindrical enclosure 25, e.g. having a substantially open top side and a substantially closed bottom side, e.g. with a bottom wall 4, as well as a circumferential wall 5. In order to drive an impeller inside the flexible container 3 (for instance the agitation device/impeller 27 as shown in FIG. 2), a drive shaft coupling 19 may be provided, connected to the flexible container 3 with a container connection 16 in the form of a bag connection 16 on the one hand and connected to a motor with a detachable motor connection 17 on the other hand, such that the impeller can be driven via the drive shaft coupling 19. For the example embodiment shown, the bioreaction process taking place in the flexible container 3 may be controlled by means of a control panel 21 and various controllers. The flexible container 3 may be mounted or arranged in the enclosure 25 of the flexible container holder 2 via a mounting opening 20, such as a door 20, e.g. a door 20 that opens sideways. The flexible container 3 may be releasably suspended from a mounting portion 6. The mounting portion 6 may be attached to the flexible container holder 2, e.g. to a top portion of the flexible container holder 2.

As shown in FIG. 2, the motor may comprise a rotatable output shaft end configured for detachable coupling to a first drive shaft end of a drive shaft 28 for driving (e.g., imparting torque and rotation to) the drive shaft 28 around a longitudinal axis X, normally a vertical axis X, in order to rotate an agitation device 27, such as an impeller 27, for mixing the fluid 14. The flexible container holder 2 as shown, may furthermore comprise a holder arm 26. The mounting portion 6 is connected to the holder arm 26. The holder arm 26 may be attached or connected to the flexible container holder 2, such as an upper portion thereof. The motor may be attached to the holder arm 26, such as an end thereof, situated on the longitudinal axis X, above the flexible container 3. The flexible container 3 may be suspended from the mounting portion 6 attached to the holder arm 26 via the bag connection 16. The agitation device 27 may comprise a three-bladed screw or the like.

As shown in FIGS. 2-7, according to the present disclosure, geometry adaptation means 7 are provided configured for adapting the geometry of the flexible container 3 with respect to the flexible container holder 2. The geometry adaptation means 7 are configured for providing the flexible container 3 with a flexible container geometry similar to a rigid multi-use bioreactor geometry during use. The geometry adaptation means 7 may more specifically be configured for providing a flexible container bottom portion 8 of the flexible container 3 with a flexible container bottom portion 8 geometry similar to a rigid multi-use bioreactor bottom portion geometry during use, as more clearly shown in FIGS. 2, 6 and 7.

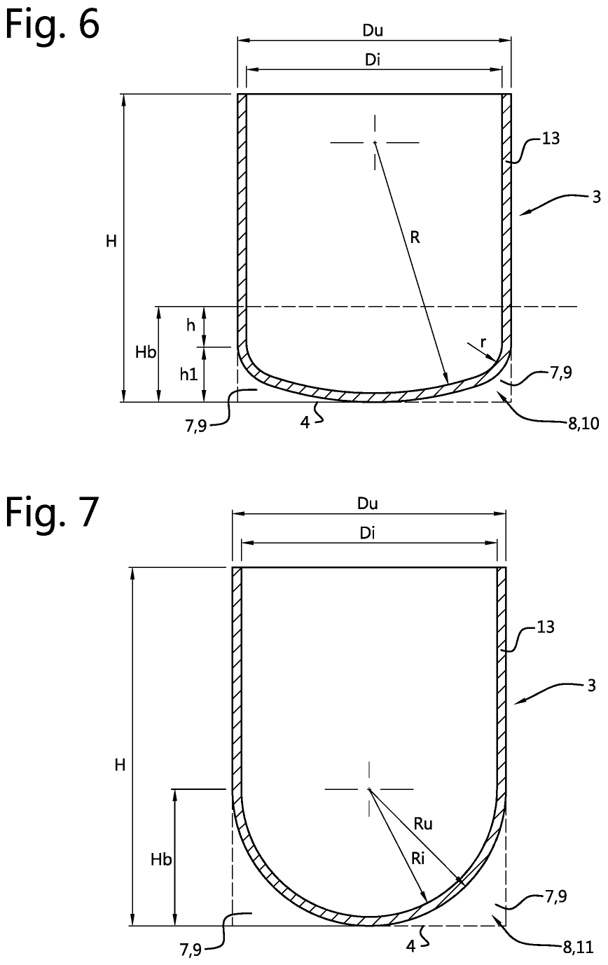

As shown in FIGS. 2 and 4-7, the geometry adaptation means 7 may comprise one or more inserts or inlays 9 arranged in the flexible container holder 2 for providing the flexible container 3 with a flexible container 3 geometry similar to a rigid multi-use bioreactor geometry (the geometry adaptation means 7 may thus not be integrally formed with the flexible container holder 2, e.g. being separately formed). More specifically, the bioreactor 1 may comprise one or more inserts 9 arranged on the bottom wall 4, between the bottom wall 4 and the flexible bottom portion 8 of the flexible container 3 (when the flexible container 3 is installed in the flexible container holder 2, i.e. during use), for providing the flexible container bottom portion 8 with a flexible container bottom portion 8 geometry similar to a rigid multi-use bioreactor bottom portion geometry during use. The one or more inserts or inlays 9 preferably have a concave top surface. The bottom surface of the one or more inserts or inlays 9 may be flat. The one or more inserts or inlays 9 arranged on the bottom wall 4 may for instance provide the flexible container bottom portion 8 with a flat, dish-shaped or hemispherical bottom portion geometry 8, as mentioned before. The one or more inserts or inlays 9 together preferably support at least 80%, more preferably at least 90%, even more preferably at least 95%, such as 100%, of the flexible container bottom portion 8 surface. The outer diameter of the configuration of the one or more inserts or inlays 9 preferably is at least 80%, more preferably at least 90%, even more preferably at least 95%, such as 100%, of the outer diameter of the flexible container 3 (i.e. Dᵤ, as shown in e.g. FIGS. 6 and 7).

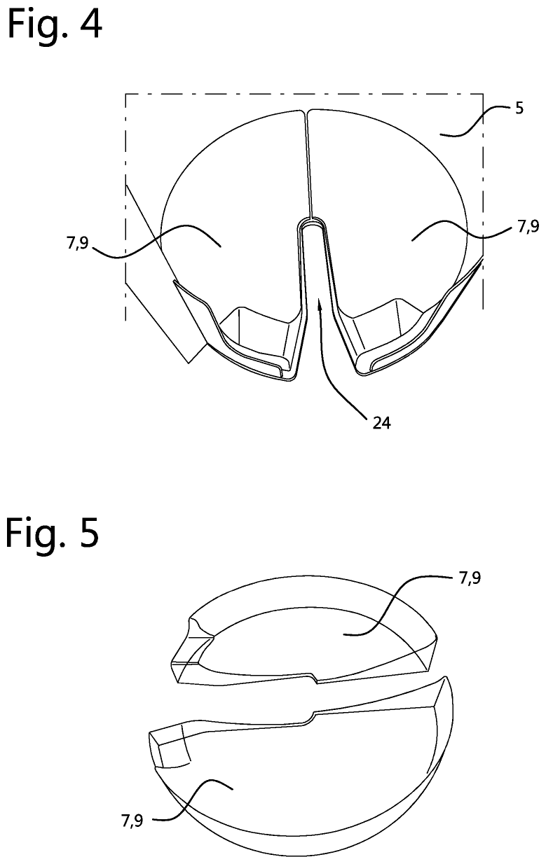

The inserts 9 may comprise one, two (as shown in FIGS. 4 and 5) or even more inserts 9. When viewed along (parallel to) the longitudinal axis X, such as from above, the inserts 9 may form or enclose a circle. When using e.g. two inserts 9, the angle which the arc of an insert 9 subtends at the centre of the circle, i.e. the central angle enclosed by the respective insert 9, may for instance be 180°—or less when a space or recess is present between adjacent inserts 9, such as the recess 24 shown in FIG. 4. The recess 24 may be tapered, i.e. decreases in width, from the outer circumference of the “circle” toward the centre thereof. The recess 24 may generally be configured for accommodating any outlets, spargers, et cetera, that may be present at the bottom side of the flexible container 3. When e.g. using three inserts 9, the central angle enclosed by an individual insert 9 may be 120° or smaller. The inserts 9 can be made of plastic or any other suitable material. The inserts 9 may e.g. be made by injection molding or 3D-printing/additive manufacturing.

As shown in FIG. 6, the geometry adaptation means 7, such as the inserts 9, may for instance be configured for providing the flexible container 3 with a flat or dish-shaped flexible container bottom portion 8, 10, having a flexible container bottom portion geometry 10 as defined on p. 5 of the present application. The outer diameter of the configuration of the one or more inserts or inlays 9 preferably is close to 100% of the outer diameter Dᵤ of the flexible container 3, as shown in FIG. 6. As mentioned on p. 5 of the present application, hi may then equal c*Dᵤ, wherein c=0.1-0.3. The height of the insert or inlay 9 at a radially outermost position of the insert or inlay 9 preferably equals the height of the flexible container bottom portion h₁ for optimal support. Purely by means of example, for a flexible container 3 having an external diameter of the bioreactor Dᵤ of e.g. 0.1-3 m, the height h₁ of the insert or inlay 9 may thus equal 0.01-0.9 m at a radially outermost position of the insert or inlay 9.

As shown in FIG. 7, the geometry adaptation means 7 may also be configured for providing the flexible container 3 with a hemispherical flexible container bottom portion 8, 11, having a flexible container geometry 11 as defined on p. 5 of the present application. Again, the outer diameter of the configuration of the one or more inserts or inlays 9 preferably is close to 100% of the outer diameter Dᵤ of the flexible container 3, as shown in FIG. 7. The height of the insert or inlay 9 at the radially outermost position thereof preferably equals the height of the flexible container bottom portion Hb for optimal support.

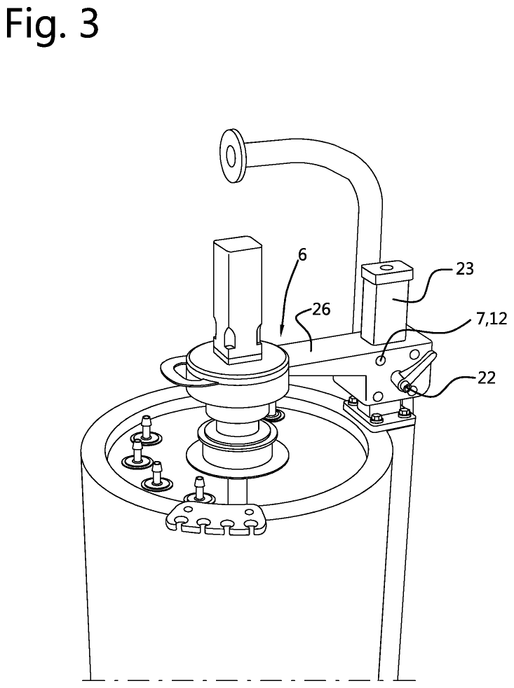

As shown in FIG. 3, the geometry adaptation means 7 may also comprise height-adjustment means 12 arranged at the mounting portion 6, for during use adjusting a height of the flexible container 3 in the flexible container holder 2, with respect to the flexible container holder 2. Thus, by adjusting the height of the flexible container 3 in the flexible container holder 2, the pressure exerted on the flexible container 3, for instance by external/separate objects, like the inserts 9, may be regulated and thus the shape of the flexible container 3 may be influenced. During filling, wrinkles at the bottom portion 8 of the flexible container 3 may be prevented or counteracted. After filling, headspace shape and volume 15 (such as shown in FIG. 2) may be adjusted by adjusting the height of the flexible container 3 in the flexible container holder 2. The height-adjustment means 12 may e.g. comprise a height-adjustment guide member 23 along which the holder arm 26 may slide upwards and downwards, in order to move the mounting portion 6 with the flexible container 3 upwards and downwards in the flexible container holder 2. A height adjustment lever 22 may be used for locking or unlocking the holder arm 26 with respect to the height adjustment guide member 23.

As mentioned before, another aspect of the disclosure concerns a method for operating a flexible container 3 in a flexible container holder 2 of an aforementioned single-use bioreactor 1, comprising the step of:
releasably mounting a flexible container 3 in the flexible container holder 2 with the mounting portion 6; and, optionally:
filling the flexible container 3 with a bioreactor fluid 14.

The method may further comprise the step of:
arranging the geometry adaptation means 7 comprising the one or more inserts 9 in the flexible container holder 2, preferably prior to filling, such that the one or more inserts 9 are arranged between the flexible container holder 2 and the flexible container 3 during use, in particular for providing the flexible container 3 with a flexible container geometry similar to a rigid multi-use bioreactor geometry.

The method may further comprise the step of:
adjusting the height of the flexible container 3 in the flexible container holder 2 with the height-adjustment means 12 during filling, in particular to prevent wrinkles from occurring at the flexible bottom portion 8 of the flexible container 3.

The height may be adjusted over a range of e.g. 0-20 cm, such as 0-10 cm, depending on the size/volume of the bioreactor 1.

The method may further comprise the step of:
adjusting the height of the flexible container 3 in the flexible container holder 2 with the height-adjustment means 12 after filling, in particular to influence headspace 15 shape and volume.

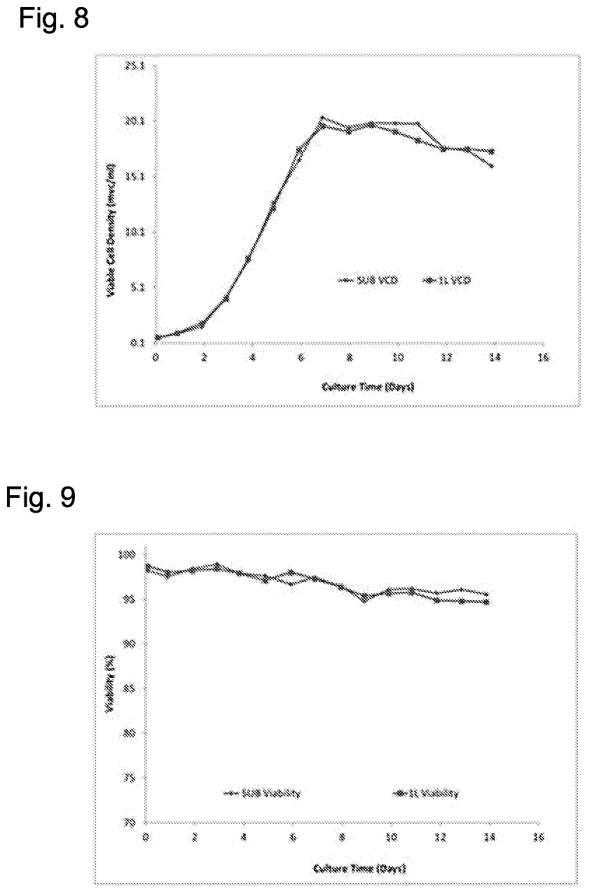

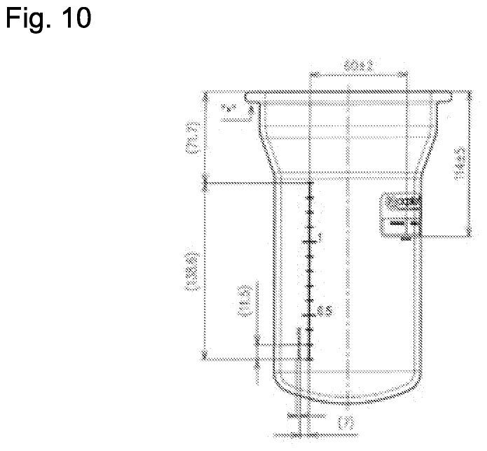

FIG. 8 shows a graph comparing visible cell density (VCD) of a cultivation in a single-use bioreactor (SUB) provided with geometry adaptation means according to the present disclosure and a cultivation in a multi-use bioreactor. VCD is plotted against culture time. A2 liter glass bioreactor (as shown in FIG. 10) was run in parallel with a 50 liter single-use bioreactor to obtain the results shown in FIGS. 8 and 9. The 2L glass had a “traditional” bioreactor shape” as shown in FIG. 10. The widening towards the neck is to allow the mounting of a 3 L top plate on the vessel. In operation, the bioreactor is filled to below the widening.

FIG. 9 shows a graph comparing cell viability of the above cultivation. Visibility is plotted against culture time.

The PVCD of the 2 liter glass bioreactor was 19.66×10⁶ on day 7 with 97.3% viability. The PVCD of the 50 liter single-use bioreactor was 20.4×10⁶ on day 7 with 97.4% viability.

Due to the use of geometry adaptation means according to the present disclosure highly similar cultivation results can thus be achieved.

Although the disclosure has been described above with reference to example embodiments, variants within the scope of the present disclosure will readily occur to those skilled in the art after reading the above description. Such variants are within the scope of the independent claims and the dependent claims. In addition, it is to be understood that express rights are requested for variants as described in the dependent claims. It should also be noted that the example embodiments shown in the Figures, or features thereof, may be combined to yield embodiments not explicitly shown in the Figures.

### LIST OF REFERENCE NUMERALS

1. Bioreactor
2. Flexible container holder
3. Flexible container
4. Flexible container holder bottom wall
5. Circumferential wall
6. Mounting portion
7. Geometry adaptation means
8. Flexible container bottom portion
9. Insert
10. Flat flexible container bottom portion
11. Hemispherical flexible container bottom portion
12. Height-adjustment means
13. Bag
14. Bioreactor fluid
15. Headspace
16. Bag connection
17. Motor connection
18. Inlet/outlet/sensor ports
19. Drive shaft coupling
20. Mounting opening
21. Control panel
22. Height adjustment lever
23. Height adjustment guide member
24. Recess between inserts
25. Enclosure
26. Holder arm
27. Impeller
28. Drive shaft

1. A bioreactor, comprising:
a flexible container holder for holding a flexible container, having a bottom wall for supporting the flexible container and a circumferential wall, extending along a longitudinal direction (X) of the flexible container holder;
a mounting portion for releasably mounting the flexible container in the flexible container holder; and
geometry adaptation means configured for adapting the geometry of the flexible container with respect to the flexible container holder,
wherein the geometry adaptation means are configured for providing the flexible container with a flexible container geometry similar to a rigid multi-use bioreactor geometry during use.

2. The bioreactor according to claim 1, wherein the geometry adaptation means are configured for providing a flexible container bottom portion of the flexible container with a flexible container bottom portion geometry similar to a rigid multi-use bioreactor bottom portion geometry during use.

3. The bioreactor according to claim 1, wherein the geometry adaptation means comprise one or more inserts arranged in the flexible container holder, between the flexible container holder and the flexible container, for providing the flexible container with a flexible container geometry similar to a rigid multi-use bioreactor geometry.

4. The bioreactor according to claim 1, wherein the geometry adaptation means comprise one or more inserts arranged on the bottom wall, between the bottom wall and the flexible bottom portion of the flexible container, for providing the flexible container bottom portion with a flexible container bottom portion geometry similar to a rigid multi-use bioreactor bottom portion geometry during use.

5. The bioreactor according to claim 1, wherein the geometry adaptation means are configured for providing the flexible container with a flat or dish-shaped flexible container bottom portion.

6. The bioreactor according to claim 5, having a flexible container geometry defined by:
$${H}_{w o r k} / {D}_{w o r k} = 2. - 2.5 , preferably 2.1 - 2.3 , and / or$$
$${V}_{w o r k} / {V}_{total} = 0.6 - 0.9 , preferably 0.6 - 0.8 , more preferably 0.65 - 0.7 ,$$
wherein:
H_{work}=working height of the bioreactor
D_{work}=working diameter of the bioreactor
V_{work}=working volume of the bioreactor
Vₜₒₜₐₗ=total volume of the bioreactor.

7. The bioreactor according to claim 5, having a flexible container bottom portion geometry defined by:
$$R = {D}_{u} , and / or$$
$${D}_{i} = {D}_{u} - 2 * {t}_{wall} , and / or$$
$$r = a * {D}_{u} , wherein a = 0.05 - 0.2 , and / or$$
$$h = b * {t}_{wall} , wherein b = 3 - 4 , and / or$$
$${h}_{1} = c * {D}_{u} , wherein c = 0.1 - 0.3 , and / or$$
$${H}_{b} = d * {D}_{u} + h , wherein d = 0.1 - 0.3 , and / or$$
$${V}_{b} = e * {D}_{i}^{3} , wherein e = 0.05 - 0.2$$
wherein:
R=radius of curvature of an inner portion of the flexible container bottom portion
Dᵢ=internal diameter of the bioreactor
Dᵤ=external diameter of the bioreactor
r=radius of curvature of an outer portion of the flexible container bottom portion
h₁=height of the flexible container bottom portion
V_{b}=volume of the flexible container bottom portion
t_{wall}=wall thickness of the bioreactor.

8. The bioreactor according to claim 1, wherein the geometry adaptation means are configured for providing the flexible container with a hemispherical flexible container bottom portion.

9. The bioreactor according to claim 8, having a flexible container geometry defined by:
$${H}_{w o r k} / {D}_{w o r k} = 0.75 - 1. , and / or$$
$${V}_{w o r k} / {V}_{total} = 0.6 - 0.9 , preferably 0.6 - 0.8 , more preferably 0.65 - 0.7 ,$$
wherein:
H_{work}=working height of the bioreactor
D_{work}=working diameter of the bioreactor
V_{work}=working volume of the bioreactor
Vₜₒₜₐₗ=total volume of the bioreactor.

10. The bioreactor according to claim 1, wherein the geometry adaptation means comprise height-adjustment means arranged at the mounting portion, for during use adjusting a height of the flexible container in the flexible container holder.

11. The bioreactor according to claim 1, wherein the flexible container is a container for a bioreaction.

12. The bioreactor according to claim 1, wherein the flexible container is configured for single use.

13. The bioreactor according to claim 1, wherein the flexible container comprises a bag.

14. The bioreactor according to claim 1, wherein the flexible container holder is configured for multi-use.

15. The bioreactor according to claim 1, comprising a flexible container arranged in the flexible container holder, wherein the mounting portion releasably mounts the flexible container in the flexible container holder.

16. The method for operating a flexible container in a flexible container holder of a bioreactor according to claim 1, comprising the step of:
releasably mounting a flexible container in the flexible container holder with the mounting portion; and, optionally:
filling the flexible container with a bioreactor fluid.

17. The method according to claim 16, further comprising:
arranging the geometry adaptation means comprising one or more inserts in the flexible container holder, such that the one or more inserts are arranged between the flexible container holder and the flexible container during use, for providing the flexible container with a flexible container geometry similar to a rigid multi-use bioreactor geometry.

18. Method according to claim 16, further comprising:
adjusting the height of the flexible container in the flexible container holder with a height-adjustment means during filling.

19. Method according to claim 16 further comprising:
adjusting the height of the flexible container in the flexible container holder with a height-adjustment means after filling.
